(19) United States
(12) Patent Application Publication
(10) Pub. No.: US 2003/0216361 A1
(43) Pub. Date: Nov. 20, 2003

(54) Therapeutic compositions effective against gram positive bacteria

(76) Inventors: George R. Pettit, Paradise Valley, AZ (US); Robin K. Pettit, Fountain Hills, AZ (US)

Correspondence Address: FENNEMORE CRAIG, 3003 N. Central Avenue, Suite 2600, Phoenix, AZ, 85012, US

(21) Appl. No.: 09/893,861

(22) Filed: Jun. 28, 2001

Related U.S. Application Data
(60) Provisional application No. 60/214,844, filed on Jun. 28, 2000.

(51) Int. Cl. A61K 31/56; A61K 31/58

(52) U.S. Cl. 514/176; 514/182

(57) ABSTRACT

The present invention relates to compounds of the formula ##STR1## and to pharmaceutically acceptable salts thereof, wherein R.sup.1 and R.sup.2 are as defined herein. The compounds are useful as anti-microbial agents, most specifically against gram positive bacteria. The invention further relates to pharmaceutical compositions and methods of treating bacterial infection using such compositions.

### CROSS-REFERENCE TO RELATED APPLICATIONS

Priority is claimed from U.S. provisional patent application Serial No. 60/214,844 filed Jun. 28, 2000 of Pettit/Pettit. That application is incorporated herein by reference.

### INTRODUCTION

1. Technical Field

The present invention relates to novel anti-microbial compositions effective against a wide spectrum of gram positive bacteria.

2. Background

There has recently been an alarming increase in resistance of gram-positive bacteria to available antimicrobials. It has been reported, for example, that many enterococci are resistant to vancomycin, that streptococci exhibit a growing resistance to penicillin and that deadly staphylococci are becoming resistant to methicillin (see, e.g. Amyes S G B, Gemmell C G. “Antibiotic resistance”, J Med Microbiol 1997;46:436-470;Pfaller M A, Jones R N, Doern G V, Kugler K and the Sentry Participants Group. “Bacterial pathogens isolated from patients with bloodstream infection: Frequencies of occurrence and anti-microbial susceptibility patterns from the SENTRY anti-microbial surveillance program (United States and Canada, 1997).” Antimicrob Agents Chemother 1998;42:1762-1770.)

The options for effective treatment are limited and novel anti-microbials to counter microorganisms resistant to accepted therapies are being sought.

3. Related art

Brooks A K, Zervos M J “New antimicrobial Agents for Gram-positive infections” Current Opinion In Infectious Diseases 1998; 11:667-671.

Doern G V, Heilmann K P, Huynh H K, Rhomberg P R, Coffman S L and Brueggemann A B, “Antimicrobial Resistance among Clinical Isolates of Streptococcus pneumoniae in the United States during 1999-2000, Including a Comparison of Resistance Rates since 1994-1995” Antimicrobial Agents and Chemotherapy 2001; 45:1721-1729.

Edwards D D, “Enterococci Attract Attention of Concerned Microbiologists” ASM News 2000; 66:540-545.

Hiramatsu K, Hideaki H. “Glycopeptide resistance in staphylococci” Current Opinion In Infectious Diseases 1998; 11:653-658.

Hunter P, “Growing Threat of Gram-positive resistance—a challenge to the industry” DDT 1997; 2: 47-49.

Paradisi F, Giampaolo C., “Treatment of Otitis Media” Current Opinion in Infectious Diseases 1998, 11:859-865.

Pettit G R, Smith R L, Klinger H. “Synthesis of 3β-Acetoxy-17βp-(L-arginyl-L-arginyl-L-prolyl)amino-5α-androstane. J Med Chem 1967; 10:145-148.

Pettit R K, Cage G D, Pettit G R, Liebman J A “Antimicrobial and cancer cell growth inhibitory activities of 3β-Acetoxy-17βp-(L-arginyl-L-arginyl-L-prolyl)amino-5α-androstane in vitro“International Journal of Antimicrobial Agents 2000;

15:299-304.

Pfaller, M A, Jones, R N, Doern, G V, Kugler, K The Sentry Participants Group “Bacterial Pathogens Isolated from Patients with Bloodstream Infection: Frequencies of Occurrence and Antimicrobial Surveillance Program (United States and Canada, 1997)” Antimicrobial Agents and Chemotherapy 1998; 1762-1770.

### SUMMARY

It has been discovered that certain androstane amides are effective as anti-microbial agents. The present invention sets forth certain androstane amides, most especially the compound 3β-acetoxy-17β-(L-prolyl)amino-5α-androstane, for preventing and controlling the growth of gram positive bacteria.

Pharmaceutical compositions comprising a therapeutically effective amount of the androstane amides, or a pharmaceutically acceptable salt thereof, and a pharmaceutically acceptable carrier are provided.

In an important aspect of the invention, methods of controlling bacterial growth and treating a bacterial infection in a mammals are provided in which a therapeutically effective amount of the present androstane amides, or a pharmaceutically acceptable salt thereof, is administered to said mammals.

### BRIEF DESCRIPTION OF THE FIGURES

The Figures illustrate the antimicrobial action of the androstane amides of the present invention against gram positive bacteria.

FIG. 1 illustrates the kill curves for S. aureus 29213 (A)with indicated multiples (MIC) of anti-microbial 3β-acetoxy-17β-(L-prolyl)amino-5α-androstane.

FIG. 2 illustrates the kill curve for E. faecalis 29212 (B) with indicated multiples (MIC) of anti-microbial 3β-acetoxy-17β-(L-prolyl)amino-5α-androstane.

FIG. 3 illustrates the kill curve for S. pneumoniae 6303 (C) with indicated multiples of anti-microbial 3β-acetoxy-17β-(L-prolyl)amino-5α-androstane.

### DETAILED DESCRIPTION OF THE INVENTION

The present invention relates to compounds of the formula (1):

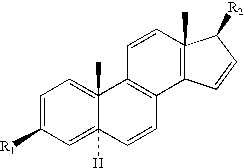

wherein R₁ is hydrogen, alkyl, alkanoyl or Y-substituted alkanoyl

wherein Y is alkyl, aryl or halo; and

R₂ is amide, or X-substituted amide wherein X is a peptide or an amino acid; or a pharmaceutically acceptable addition salt and/or hydrate thereof, or where applicable, a geometric or optical isomer or racemic mixture thereof.

Preferably R₁ is alkanoyl, most preferably acetyl or propyl and R₂ is amide comprising proline. Also presented are those closely related analogues of 3β-acetoxy-17β-(L-prolyl)amino-5α-androstane which may be prepared by substituting the hydrogen atoms on the androstane cyclic backbone by one or more chemical groups selected from the group comprising alkyl aryl, alkoxyl and halo by methods known in the art.

The preferred embodiment comprises a compound having the formula 3β-acetoxy-17β-(L-prolyl)amino-5α-androstane.

The term “alkyl”, as used herein, unless otherwise indicated, includes saturated monovalent hydrocarbon radicals having straight, cyclic or branched moieties. Said alkyl group may include one or two double or triple bonds. It is understood that for cyclic moieties at least three carbon atoms are required in said alkyl group.

The term “alkanoyl”, as used herein, unless otherwise indicated, includes —OC(O)-alkyl groups wherein “alkyl” is as defined above.

The term “aryl”, as used herein, unless otherwise indicated, includes an organic radical derived from an aromatic hydrocarbon by removal of one hydrogen, such as phenyl or naphthyl.

The term “amide” as used herein, unless otherwise indicated, includes —NC(O)— and substituted amide includes alkyl, aryl and the C-terminus end of an amino acid or peptide. Generally the term “amide” includes amino acid or peptide groups bound to the 17β position of the androstane molecule by means of a linkage through the terminal nitrogen group.

The present compounds are effective bactericides against gram-positive bacteria and are therefore useful therapeutic agents for treatment of diseases caused by these bacteria. The term “treatment”, as used herein, unless otherwise indicated, includes the treatment or prevention of bacterial growth and infection as provided in the method of the present invention.

As used herein, unless otherwise indicated, the term “bacterial infection(s)” includes bacterial infections that occur in mammals as well as disorders related to bacterial infections that may be treated or prevented by administering antibiotics such as the compounds of the present invention. Such bacterial infections and disorders related to such infections are represented by the following: pneumonia, otitis media, sinusitus, bronchitis, tonsillitis, and mastoiditis, pharynigitis, rheumatic fever, and glomerulonephritis related to uncomplicated skin and soft tissue infections, abscesses and osteomyelitis, and puerperal fever and uncomplicated acute urinary tract infections related to infection by gram positive bacteria. Other bacterial infections and disorders related to such infections may be treated or prevented in accordance with the method of the present invention.

As is illustrated in Table 2, the following gram positive bacteria may be treated by the present compounds: Methicillin-resistant Staphylococcus aureus, Staphylococcus saprophyticus, Vancomycin-resistant Enterococcus spp., Vancomycin-resistant Enterococcus faecalis, Vancomycin-resistant Enterococcus faecium, Penicillin-resistant Streptococcus pneumoniae, invasive Streptococcus pneumoniae, Group A Streptococcus, Bacillus subtilis, Bacillus cereus, Bacillus circulans, Bacillus licheniformis, Paenibacillus alvei, Rhodococcus spp., Rhodococcus equi, Gordona bronchialis, Gordona sputi, Listeria monocytogenes, cornybacterium diphtheriae, nocardia asteroides, Norcardia farcinica, lactobacillus spp., arcanobacterium haemolyticum and gardnerella vaginalis. As illustrated in Table 1, the following organisms may also be treated by compounds of the present invention: Staphylococcus aureus, staphylococcus epidermidis, Enterococcus faecalis, Streptococcus pneumoniae, Micrococcus luteus and Bacillus subtilis.

Certain compounds of formula 1 may have asymmetric centers and therefore exist in different enantiomeric forms. This invention relates to the use of all optical isomers and stereoisomers of the compounds of formula 1 and mixtures thereof. The compounds of formula 1 may also exist as tautomers. This invention relates to the use of all such tautomers and mixtures thereof.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The present invention also includes all radiolabelled forms of the compounds of formula 1, and pharmaceutically acceptable salts thereof, wherein the radiolabel is selected from ³H, ¹¹C ¹⁴C. Such radiolabelled compounds are useful as research or diagnostic tools.

The preferred embodiment of compounds of the present invention can be prepared as described by Pettit G R et al. in: Pettit G R, Smith R L, Klinger H. “Synthesis of 3β-Acetoxy-17bp-(L-arginyl-L-arginyl-L-prolyl)amino-5a-androstane” J. Med. Chem. 10 145-148 (1967), which is herein incorporated by reference in its entirety. Certain other derivatives may likewise be prepared by similar methods to make the compounds of the present invention.

The present invention also includes pharmaceutically acceptable salts and derivatives of the compounds of the invention.

The phrase “pharmaceutically acceptable salt(s)”, as used herein, unless otherwise indicated, includes salts of acidic or basic groups which may be present in the compounds of formula 1. Salt formation may be possible when one of the substituents carries an acidic or basic group. Salts may be prepared by salt exchange in conventional manner.

The compounds of formula 1 that are basic in nature are capable of forming a wide variety of salts with various inorganic and organic acids. The acids that may be used to prepare pharmaceutically acceptable acid addition salts of such basic compounds of formula 1 are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the formula 1 that are acidic in nature, are capable of forming base salts with various pharmacologically acceptable cations. Examples of such salts include the alkali metal or alkaline-earth metal salts and particularly, the sodium and potassium salts. These salts may be prepared by conventional techniques. The chemical bases which are used as reagents to prepare the pharmaceutically acceptable base salts of this invention are those which form non-toxic base salts with the acidic compounds of formula 1. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of this invention may be in crystalline or non-crystalline form, and, if crystalline, may optionally be hydrated or solvated. When some of the compounds of this invention are allowed to crystallize or are re-crystallized from organic solvents, solvent of crystallization may be present in the crystalline product. This invention includes within its scope such solvates. Similarly, some of the compounds of this invention may be crystallized or re-crystallized from solvents containing water. In such cases water of hydration may be present in the crystalline product. This invention includes within its scope stoichiometric hydrates as well as compounds containing variable amounts of water that may be produced by processes such as lyophilization.

The compounds according to the invention are suitably provided in substantially pure form, for example at least 50% pure, suitable at least 60% pure, advantageously at least 75% pure, preferably at least 85% pure, more preferably at least 95% pure, especially at least 98% pure, all percentages being calculated as weight/weight. An impure or less pure form of a compound according to the invention may, for example, be used in the preparation of a more pure form of the same compound or of a related compound (for example a corresponding derivative) suitable for pharmaceutical use.

The compounds of the present invention and their pharmaceutically acceptable salts or derivatives have antimicrobial properties and are useful for the treatment of microbial infections in animals, especially mammals. The compounds may be used for the treatment of infections caused by Gram-positive bacteria, including, for example, Methicillin-resistant Staphylococcus aureus, Staphylococcus saprophyticus, Vancomycin-resistant Enterococcus spp., Vancomycin-resistant Enterococcus faecalis, Vancomycin-resistant Enterococcus faecium, Penicillin-resistant Streptococcus pneumoniae, invasive Streptococcus pneumoniae, Group A Streptococcus, Bacillus subtilis, Bacillus cereus, Bacillus circulans, Bacillus licheniformis, Paenibacillus alvei, Rhodococcus spp., Rhodococcus equi, Gordona bronchialis, Gordona sputi, Listeria monocytogenes, cornybacterium diphtheriae, nocardia asteroides, Norcardia farcinica, lactobacillus spp., arcanobacterium haemolyticum and gardnerella vaginalis staphylococcus epidermidis, Enterococcus faecalis, Streptococcus pneumoniae, Micrococcus luteus and Bacillus subtilis.

In an important aspect of the invention, a pharmaceutical composition is provided which comprises a compound of formula 1 or a pharmaceutically acceptable salt or derivative thereof together with a pharmaceutically acceptable carrier or excipient. The compounds and compositions according to the invention may be formulated for administration in any convenient way for use in human or veterinary medicine, by analogy with other antibiotics.

The compounds and compositions according to the invention may be formulated for administration by any route, for example oral, topical or parenteral. Suitable pharmaceutical carriers include inert solid diluents or fillers, sterile aqueous solutions and various organic solvents. Most preferably, the compositions are formulated for administration by topical means.

The compounds of formula 1 and their pharmaceutically acceptable salts (hereinafter referred to, collectively, as “the active compounds of this invention”) may be administered alone or in combination with pharmaceutically acceptable carriers, in either single or multiple doses. In certain preferred embodiments, the carrier comprises salts and buffers or other suitable means for controlling the pH of the topical composition to a value that physiologically compatible for the patient, yet at a value that enhances the bactericidal activity of the active compounds.

Compositions according to the invention intended for topical administration may, for example, be in the form of ointments, creams, lotions, eye ointments, eye drops, car drops, nose drops, nasal sprays, impregnated dressings, and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

The invention further provides the use of a compound of the invention or a pharmaceutically acceptable salt or derivative thereof in the preparation of a medicament composition for use in the treatment of microbial infections.

In another important aspect of the present invention, a method is provided for treating microbial infections in mammals including humans and domesticated humans, which comprises administering an anti-microbial-effective amount of compound of formula 1 or a pharmaceutically acceptable salt or derivative thereof, or a composition according to the invention, to a patient in need thereof.

To implement the methods of this invention, an effective dose of an active compound of this invention is administered to a susceptible or infected mammal by topical application to the skin and/or mucous membranes. The route of administration will depend on the mammals that is being treated. The effective dose will vary with the severity of the infection. In single dose murine toxicity evaluations, the androstane derivatives were non-toxic at doses up to 400 mg/kg as reported in Pettit R K, Cage G D, Pettit G R, Liebman J A “Antimicrobial and cancer cell growth inhibitory activities of 3β-Acetoxy-17βp-(L-arginyl-L-arginyl-L-prolyl)amino-5α-androstane in vitro” International Journal of Antimicrobial Agents 2000; 15: 299-304, which is hereby incorporated in its entirety by reference. The upper limit of safe and effective doses is thus very high and is up to about 1 gram/kg body weight. When applied topically from a methanol solution, the compound is effective over a period of days at a dose of about 25 to 50 mg/kg (see, e.g. Example 6).

Topical preparations may be administered by one or more applications per day to the affected area; over skin areas occlusive dressings may advantageously be used. Continuous or prolonged delivery may be achieved by an adhesive reservoir system. The individual compounds of such combinations may be administered either sequentially or simultaneously in separate or combined pharmaceutical formulations. Appropriate doses of known therapeutic agents will be readily appreciated by those skilled in the art.

In another important aspect of the present invention, a method of inhibiting the growth of gram positive bacteria is presented. In the method the bacteria are contacted with certain active compounds of this invention. Gram positive bacteria that are penicillin-resistant are especially important subjects of the anti-microbial compounds.

The Examples provided below illustrate specific embodiments of the invention, but the invention is not limited in scope to the Examples specifically exemplified.

Experimental Section

The activity of the compounds of the present invention against gram positive bacteria is demonstrated by the compound's ability to inhibit growth of defined strains of human gram positive pathogens. The following experiments were performed to illustrate the growth characteristics of a chosen panel of pathogenic gram positive bacteria in the presence of certain anti-microbial compounds of the present invention.

Methods

Disk diffusion susceptibility testing. Disk assays were performed according to National Committee for Clinical Laboratory Standards (NCCLS) (National Committee for Clinical Laboratory Standards. Performance Standards for Antimicrobial Disk Susceptibility Tests: Approved Standard M2:A6, Wayne, Pa.: MCCLS, 1997) on clinical isolates of selected gram positive bacteria and reference standards.

MIC was defined as the lowest drug concentration resulting in no visible growth of the test organism (optically clear).

Broth macrodilution susceptibility testing. Androstane amides were screened against reference strains and clinical isolates by the NCCLS broth macrodilution assay as described in “National Committee for Clinical Laboratory Standards. Methods for dilution antimicrobial susceptibility tests for bacteria that grow aerobically”; Approved standard M7-A4. NCCLS, Wayne, Pa., 1997.

Minimum bactericidal concentrations (MBC) were determined by subculturing 0.1 ml from each tube with no visible growth in the MIC broth macrodilution series onto drug-free plates. The plates were incubated at the appropriate temperature for 24-48 h.

MBC was defined as the lowest drug concentration that resulted in a ≧99.9% reduction in the initial inoculum.

In the following Examples, the source of reagents was as follows:

Anti-microbial 3β-acetoxy-17β-(L-prolyl)amino-5α-androstane. The androstane derivative (Formula 1) was synthesized according to the method of Pettit et al. as previously described hereinabove, stored desiccated, and prior to each assay, suspended in a small volume of methanol (MeOH) and diluted in the appropriate broth.

Bacterial strains tested. Non-duplicate clinical isolates and antibiotic resistance information were obtained from the Arizona Department of Health Services. Invasive Streptococcus pneumoniae were cultured from sterile sites; their antibiotic resistance profiles have not been determined. Reference strains were obtained from the American Type Culture Collection (Rockville, Md.) or Presque Isle Cultures (Presque Isle, Me.).

### EXAMPLE 1

The antimicrobal activities of 3β-acetoxy-17β-(L-prolyl)amino-5α-androstane with certain pathogenic gram positive bacteria were studied with the disk diffusion assay. Disk diffusion susceptibility testing was performed with the following:

Mueller-Hinton agar supplemented with 5% sheep blood was used for S. pneumoniae, gonococcal typing agar for Neisseria gonorrhoeae Mueller-Hinton agar for all other bacteria

Results are given in Table 1.

TABLE 1
Antimicrobial activities of 3β-acetoxy-17β-
(L-prolyl)amino-5α-androstane for reference strains in the disk diffusion
assay

| | ATCC | |
|---|---|---|
| Organism | (or Presque Isle) no. | MIC (μg/disk) |
| Staphylococcus aureus | 29213 | 12.5-25 |
| Staphylococcus epidermidis | (4653) | 6.25-12.5 |
| Enterococcus faecalis | 29212 | 25-50 |
| Streptococcus pneumoniae | 6303 | 50-100 |
| Micrococcus luteus | (456) | 1.56-3.12 |
| Bacillus subtilis | (620) | 3.12-6.25 |
| Stenotrophomonas maltophilia | 13637 | >100 |
| Pseudomonas aeruginosa | (99) | >100 |
| Escherichia coli | 25922 | >100 |
| Neisseria gonorrhoeae | 49226 | >100 |
| Enterobacter cloacae | 13047 | >100 |
| Klebsiella pneumoniae | (344) | >100 |
| Proteus vulgaris | (365) | >100 |
| Candida albicans | 90028 | >100 |
| Cryptococcus neoformans | 90112 | >100 |

### EXAMPLE 2

This experiment illustrates a study of the time required (time-kill studies) for anti-microbial action of 3β-acetoxy-17β-(L-prolyl) amino-5α-androstane with certain gram positive bacteria.

Overnight cultures of Staphylococcus aureus 29213, Enterococcus faecalis 29212 and S. pneumoniae 6303 in MHII broth were inoculated into the same medium containing multiples of the broth macrodilution MIC of 3β-acetoxy-17β-(L-prolyl) amino-5α-androstane, or an equivalent volume of MeOH. Cultures were shaken at 37° C., and aliquots were aseptically removed at various times for dilution plating. Standard errors of the means were calculated from at least two experiments. The detection limit in these experiments was 10 CFU/ml.

Observations: Killing was time-dependent for S. aureus, E. faecalis and S. pneumoniae, and concentration-dependent for S. pneumoniae. For S. aureus, time to 99.9% kill was between 8 and 24 h at sixteen and thirty-two times the MIC. For E. faecalis, time to 99.9% kill was 8 h at two and four times the MIC, and 4 h at eight times the MIC. For S. pneumoniae, time to 99.9% kill was 6 h at the MIC and 2 h at two times the MIC. The number of survivors in cultures of S. aureus, E. faecalis and S. pneumoniae treated with intermediate doses for 24 h varied greatly (note large standard errors at t=24 h for S. aureus treated with eight times the MIC, E. faecalis treated with two times the MIC, and S. pneumoniae treated with one-half the MIC). After 24 h, there were no survivors in E. faecalis cultures treated with four or eight times the MIC, and S. pneumoniae cultures treated with one and two times the MIC.

Results:

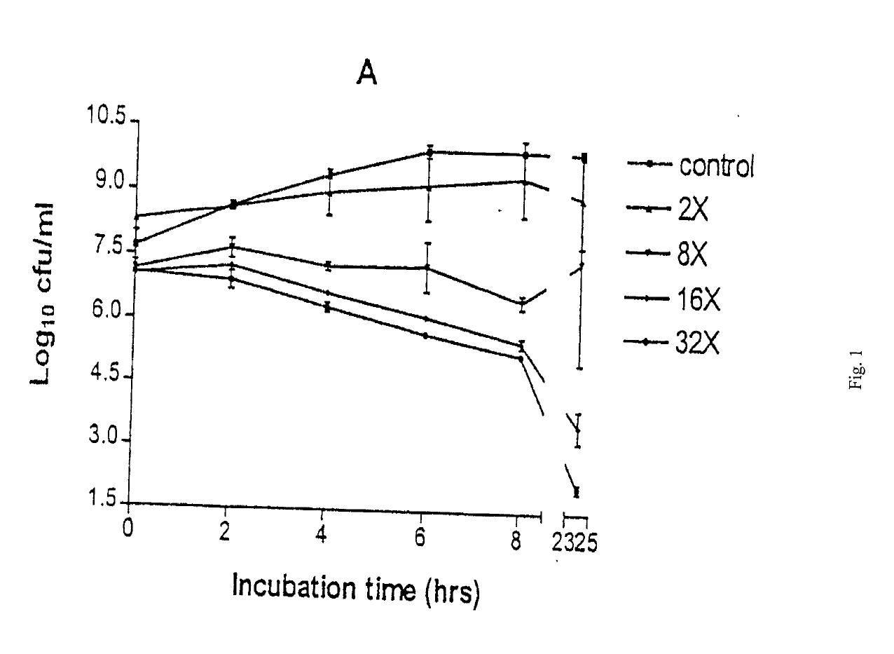

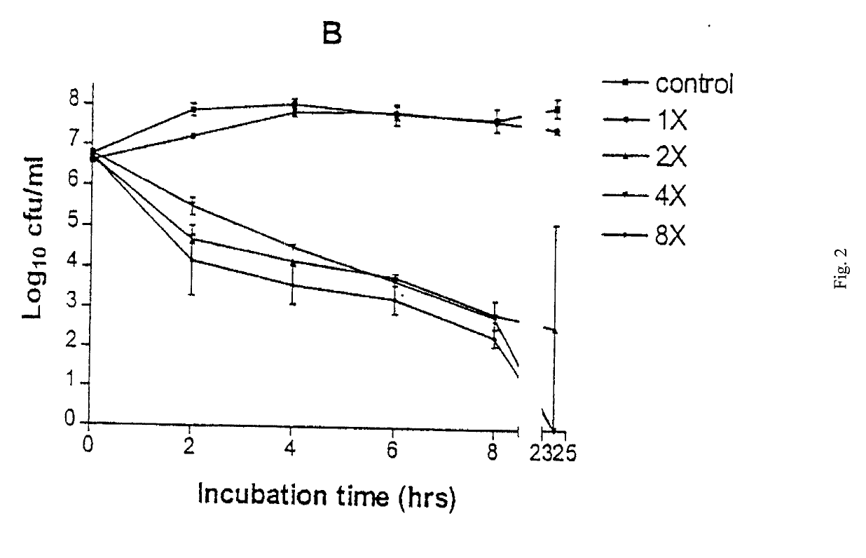

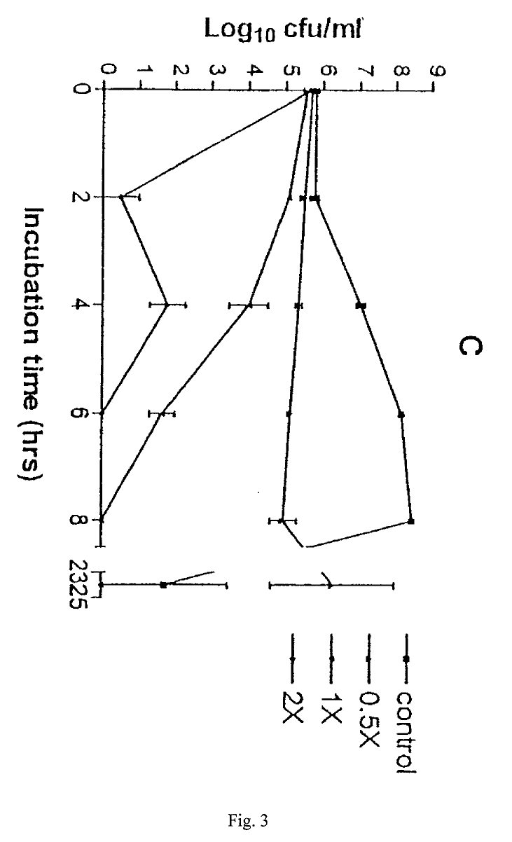

FIGS. 1-3 illustrate the time-kill curves for 3β-acetoxy-17β-(L-prolyl)amino-5α-androstane with S. aureus 29213 (FIG. 1), E. faecalis 29212 (FIG. 2) and S. pneumoniae 6303 (FIG. 3).

### EXAMPLE 3

This example illustrates broth macrodilution susceptibility testing with 3β-acetoxy-17β(L-prolyl)amino-5α-androstane drug against reference strains and certain clinical isolates by the NCCLS broth macrodilution assay. Isolated colonies from overnight cultures were suspended and diluted, as recommended, to yield final inocula of approximately 5×10⁵ CFU/ml. Tests were performed in sterile plastic tubes (12 by 75 mm) containing twofold dilutions of the androstane derivative in Mueller Hinton II (MHII) (cation adjusted) broth containing 3% lysed horse blood (Streptococcus, Arcanobacterium, Lactobacillus, Gardnerella) or MHII broth (all other bacteria tested). One tube was left drug-free (but contained an equivalent volume of MeOH) for a turbidity control. Tubes were incubated without agitation at 37° with 5% CO₂ (Streptococcus, Arcanobacterium, Lactobacillus, Gardnerella), at 37° C. (Staphylococcus, Enterococcus) or at 35° C. (Bacillus, Paenibacillus, Rhodococcus, Gordona, Micrococcus, Listeria, Corynebacterium, Nocardia).

MICs were determined after 24 h for all organisms except Gardnerella and Rhodococcus, which were read after 48 h, and Gordona sputi, which was read at 72 h.

Observations:

In these broth macrodilution assays, 3β-acetoxy-17β-(L-prolyl) amino-5α-androstane inhibited the growth of all gram-positive bacteria tested, including those resistant to methicillin, vancomycin and penicillin (Tables 2 and 3). MBC/MIC ratios were ≦2 for 73% of methicillin-resistant S. aureus, 59% of vancomycin-resistant Enterococcus spp., 88% of penicillin-resistant S. pneumoniae, 93% of invasive S. pneumoniae, 89% of Group A Streptococcus and 58% of Rhodococcus spp., consistent with a bactericidal mechanism of action. Given that the majority of bacterial pathogens isolated from cancer patients are gram-positive, the dual biological activities of this compound are noteworthy ( Koll B S, Brown A E. “The changing epidemiology of infections at cancer hospitals” Clin Infect Dis 1993; 17(Suppl. 2):S322-328).

Results are given in Tables 2 and 3 which illustrate MICs and MBCs of clinical isolates and reference strains, respectively.

TABLE 2
Broth macrodilution MICs and MBCs of
3β-acetoxy-17β-(L-prolyl)amino-5α-androstane for clinical isolates

| | | MIC (μg/ml) | | MBC (μg/ml) | | |
|---|---|---|---|---|---|---|
| Organism (no. of strains) | Range | 50%^{a} | 90%^{a} | Range | 50%^{b} | 90%^{b} |
| Methicillin-resistant | 4-8 | 8 | 8 | 8->64 | 16 | >64 |
| Staphylococcus aureus (22) | | | | | | |
| S. saprophyticus (3) | 4-8 | | | 8 | | |
| Vancomycin-resistant | 4-16 | 8 | 16 | 8->64 | 16 | >64 |
| Enterococcus spp. (34) | | | | | | |
| Vancomycin-resistant | 8-16 | | | 16-64 | | |
| E. faecalis (2) | | | | | | |
| Vancomycin-resistant | 8-16 | | | 16-64 | | |
| E. faecium (2) | | | | | | |
| Penicillin-resistant | | | | | | |
| Streptococcus pneumoniae (35) | 8-32 | 16 | 16 | 8->64 | 16 | 32 |
| Invasive S. pneumoniae (15) | 8-16 | 8 | 16 | 8-64 | 16 | 16 |
| Group A Streptococcus (18) | 8-16 | 8 | 16 | 8-32 | 16 | 32 |
| Bacillus subtilis (4) | 8 | | | 8->64 | | |
| B. cereus (5) | 32 | | | >64 | | |
| B. circulans (1) | 16 | | | >64 | | |
| B. licheniformis (1) | 32 | | | >64 | | |
| Paenibacillus alvei (1) | 16 | | | >64 | | |
| Rhodococcus spp. (19) | 4-64 | 8 | 16 | 8->64 | 16 | 32 |
| R. equi (3) | 4-8 | | | 16-32 | | |
| Gordona bronchialis (1) | 8 | | | 8 | | |
| C. sputi (1) | 8 | | | 32 | | |
| Listeria monocytogenes (3) | 16-32 | | | 32-64 | | |
| Corynebacterium diphtheriae (3) | 4-8 | | | 8 | | |
| Nocardia asteroides (1) | 8 | | | 32 | | |
| N. farcinica (1) | 16 | | | 64 | | |
| Lactobacillus spp. (1) | 16 | | | 32 | | |
| Arcanobacterium haemolyticum (1) | 16 | | | 32 | | |
| Gardnerella vaginalis (2) | 4 | | | 8 | | |

^{a}50% and 90%, MICs at which 50 and 90% of the strains, respectively, are inhibited.
^{b}50% and 90%, MBCs at which 50% and 90% of the strains, respectively, are killed.

TABLE 3
Broth macrodilution MICs and MBCs of 3β-acetoxy-17β-
(L-prolyl)amino-5α-androstane for reference stains

| | ATCC | MIC | MBC |
|---|---|---|---|
| Organism | (or Presque Isle) no. | (μg/ml) | (μg/ml) |
| Staphylococcus aureus | 29213 | 4 | 64 |
| S. epidermidis | (4653) | 8 | 32 |
| Enterococcus faecalis | 29212 | 16 | 32 |
| Streptococcus pneumoniae | 6303 | 16 | 16 |
| Bacillus subtilis | (620) | 4 | 4 |
| Micrococcus luteus | (456) | 8 | 64 |
| Corynebacterium hoagi | 7005 | 8 | 32 |

### EXAMPLE 4

This example illustrates the effect of pH on MICs of the strains tested in Example 3. Broth macrodilution assays were also performed on three separate days in MHII broth prepared at pH 6, pH 7 and pH 8. Minimum bactericidal concentrations were determined by subculturing 0.1 ml from each tube with no visible growth in the MIC broth macrodilution series onto drug-free plates. The plates were incubated at the appropriate temperature for 24-48 h. The MICs were usually within two, 2-fold dilutions. Colonies growing on drug-containing agar plates were considered resistant.

Observations: There were no survivors on plates containing eight times the MIC. Results are given in Table 4.

TABLE 4
Effect of pH on broth macrodilution MICs (range of 3 determinations)
of 3β-acetoxy-17β-(L-prolyl)amino-5α-
androstane for reference strains and clinical isolates

| | | MIC |
|---|---|---|
| Organism | pH | range (μg/ml) |
| Methicillin-resistant | 6 | 16 |
| Staphylococcus aureus^{a} | 7 | 8 |
| | 8 | 4 |
| S. aureus ATCC 29213 | 6 | 8-16 |
| | 7 | 8 |
| | 8 | 4 |
| Vancomycin-resistant | 6 | 32 |
| Enterococcus faecalis^{a} | 7 | 8 |
| | 8 | 4-8 |
| E. faecalis ATCC 29212 | 6 | 32 |
| | 7 | 8-16 |
| | 8 | 4-8 |
| Bacillus subtilis | 6 | 8 |
| Presque Isle 620 | 7 | 4-8 |
| | 8 | 4-8 |
| Listeria monocytogenes^{a} | 6 | 32 |
| | 7 | 16-32 |
| | 8 | 16 |
| Corynebacterium diphtheriae^{a} | 6 | 16 |
| | 7 | 4-8 |
| | 8 | 2-4 |

^{a}Clinical isolate

### EXAMPLE 5

This experiment illustrates the frequency of spontaneous mutants in the presence of 3β-acetoxy-17β-(L-prolyl)amino-5α-androstane.

Overnight cultures of S. aureus 29213, E. faecalis 29212, S. pneumoniae 6303 and Bacillus subtilis 620 were diluted to an OD₆₂₅ₙₘ=0.08 0.1 ml of each preparation was spread onto agar plates containing four or eight times the broth macrodilution MIC of the androstane derivative. The starting inoculum for each organism was also diluted and plated onto drug-free plates for determination of CFU/ml. After a 24 h incubation at the appropriate temperature, the number of bacterial colonies on drug-supplemented agar was counted. The frequency of occurrence of spontaneous mutants was calculated by dividing the number of colonies on drug containing plates by the number of colonies in the inoculum. When no colonies were visualized on drug-containing plates, the calculation was (<) 1 colony divided by the number of colonies in the inoculum.

Results: The frequency of occurrence of spontaneous mutants resistant to 3β-acetoxy-17β-(L-prolyl)amino-5α-androstane is given in Table 5.

TABLE 5
Frequency of occurrence of spontaneous mutants
resistant to 3β-acetoxy-17β-(L-prolyl)amino-5α-androstane

| | ATCC (or | Frequency | Frequency |
|---|---|---|---|
| Organism | Preseque Isle) no. | at 4X MIC | at 8X MIC |
| Staphylococcus aureus | 29213 | 1.2 × 10⁻⁶ | <6 × 10⁻⁹ |
| Enterococcus faecalis | 29212 | 8.9 × 10⁻⁷ | <1 × 10⁻⁸ |
| Streptococcus | 6303 | <3.3 × 10⁻⁶ | <3.3 × 10⁻⁶ |
| pneumoniae | | | |
| Bacillus subtilis | (620) | <2.2 × 10⁻⁷ | <2.2 × 10⁻⁷ |

### EXAMPLE 6

This example illustrates the effective bactericidal action of 3β-acetoxy-17β-(L-prolyl)amino-5α-androstane (herein referred to as GRP-B-10-15A or GRPB) in the mouse model of topical Gram positive (Staphylococcus aureus) infection. The model, as described in J. Gisby and J. Bryant, Antimicrobial Agents and Chemotherapy, February 2000, Vol. 44, No. 2, p 255-260, was used.

Fifty HSD:ICR male mice weighing 12-14 grams were ordered and allowed to acclimate 7 days. Mice were allocated to 5 groups of 10 animals each.

Group 1: infected suture placed, no treatment

Group 2: topical GRPB, 12.5 mg/kg BID×7 days

Group 3: topical GRPB, 25 mg/kg BID×7 days

Group 4: clean suture, no treatment

Group 5: enrofloxacin (Baytril) 10 mg/kg SQ SID×7 days

ATCC 14154, mouse virulent Staphylococcus aureus was grown in BHI broth overnight (12 hours) at 38 degrees C. Forty-five minutes prior to surgery, silk suture (2-0 size, 1 cm lengths w/swaged-on blunt needle) was soaked in the broth for 30 minutes, excess liquid was then removed by blotting with filter paper.

Mice were anesthetized with a ketamine (40 mg/kg), midazolam (2 mg/kg), and butorphanol (0.1 mg/kg) as a single IP injection.

The skin on the back of the mouse was prepared for aseptic surgery by clipping, cleansing with iodine scrub, and rinsing with alcohol. Mice were kept warm during surgery on a recirculating hot water blanket.

The suture was placed just under the skin on the dorsum of the animal using sterile instruments. The suture was knotted in the subcutaneous tissue to keep it in place. A half-thickness skin wound was made over the suture using the side of an 18 gauge needle as a scalpel.

Mice were allowed to recover in the home cage.

GRPB-10-15A was reconstituted as directed, using a minimal amount of methanol followed by dilution to required volume in sterile physiologic saline.

Injections were given using insulin syringes with 30 gauge needles. Topical administration was placed over the wound site. The mice were first treated 4 hours after surgery and were treated once or twice daily thereafter.

After 7 days of treatment animals were euthanized with IP pentobarbital euthanasia solution. The suture lengths were removed and placed in 2 mls phosphate buffered saline. Serial dilutions were performed and results reported as actual counts (number of Staph colonies per suture).

Results:

Total colonies

| | Group 1 (no | 2 (low | 3(high | 4(no | |
|---|---|---|---|---|---|
| | treatment) | GRPB) | GRPB) | infection) | 5(Baytril) |
| Animal | | | | | |
| 1 | 8940000 | 1174 | 626 | 4 | 0 |
| 2 | 1150000 | 399 | 572 | 17 | 74 |
| 3 | 2130000 | 2523 | 456 | 3 | 13 |
| 4 | 1920000 | 968 | 344 | 0 | 0 |
| 5 | 980000 | 739 | 565 | 0 | 740 |
| 6 | 2310000 | 1109 | 362 | 25 | 22 |
| 7 | 6700000 | 227 | 890* | 7 | 18 |
| 8 | 7560000 | 1993 | 328 | 5 | 30 |
| 9 | 9800000 | 832 | 483 | 32 | 24 |
| 10 | 5630000 | 317 | 289 | 7 | 100 |
| Ave. | 4712000 | 1028 | 492 | 10 | 102 |

*Mouse removed suture on last day, culture was from suture after it was on bottom of cage for at least 2 hours.

Discussion:

All animals remained healthy and active throughout this study. All mice weighed between 23 and 25 grams at the end of the study. There were no adverse clinical effects noticed.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

We claim:

1. A pharmaceutical composition for the treatment of a bacterial infection in a mammal which comprises a therapeutically effective amount of a compound having the formula 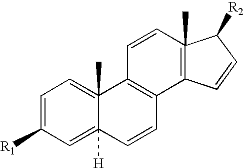
wherein:
R₁ is hydrogen, alkyl, alkanoyl or Y-substituted alkanoyl
wherein Y is alkyl, aryl or halo; and
R₂ is amide, or X-substituted amide wherein X is a peptide or an amino acid; or a pharmaceutically acceptable addition salt and/or hydrate thereof, or where applicable, a geometric or optical isomer or racemic mixture thereof.

2. The pharmaceutical composition of claim 1 wherein R₁ is alkanoyl and R₂ is X-substituted amide wherein X is an amino acid residue.

3. The pharmaceutical composition of claim 1 wherein R₁ is acetyl and R₂ is prolyl.

4. The pharmaceutical composition of claim 1 wherein said compound has the formula: 3β-acetoxy-17β-(L-prolyl)amino-5α-androstane.

5. The pharmaceutical composition of claim 1 and a pharmaceutically acceptable carrier.

6. A pharmaceutical composition according to claim 5 in a form suitable for topical administration.

7. A pharmaceutical composition according to claim 5 wherein said carrier is selected from the group comprising lotion, salve, ointment, cream or oil.

8. A pharmaceutical composition according to claim 1 comprising in addition a second anti-microbial agent.

9. A pharmaceutical composition according to claim 5 comprising in addition means for controlling the pH of said composition.

10. A method of treating a gram positive bacterial infection in a mammal which comprises administering to said mammal an antimicrobial-effective amount of a compound of claim 5.

11. The method of claim 10 wherein said antimicrobial-effective amount is between about 25 milligram to about 1 gram per kilogram body weight of said mammal treated.

12. A method of inhibiting the growth of gram positive bacteria comprising contacting said bacteria with a compound of claim 1.

13. The method of claim 10 wherein said gram-positive bacteria are selected from the group comprising penicillin-resistant, methicillin-resistant and vancomycin resistant gram-positive bacteria.

14. The method of claim 10 wherein said compound is administered to said mammal by topical means.

15. The method of claim 14 wherein said means is selected from the group comprising lotion, oil, emulsion and creme.

16. The method of claim 14 wherein said means comprises a surface-adhering dressing impregnated with said compound.
